(19) United States
(12) Patent Application Publication
(10) Pub. No.: US 2003/0023149 A1
(43) Pub. Date: Jan. 30, 2003

(54) Microfluidic microorganism detection system

(76) Inventors: Carlo D. Montemagno, Los Angeles, CA (US); Hercules Neves, Moorpark, CA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/187,309

(22) Filed: Jul. 1, 2002

Related U.S. Application Data
(60) Provisional application No. 60/302,273, filed on Jun. 29, 2001.

(51) Int. Cl. A61B 5/00

(52) U.S. Cl. 600/300

(57) ABSTRACT

A system and method for detecting microorganisms and abiotic or biotic contaminants in fluids, including food and potable and environmental waters. Various embodiments of the system include a capillary transport element and a microsensor element. The capillary transport element isolates and purifies the targeted substance. The microsensor element includes a channel with electrodes for detecting dielectric properties of the targeted substance. Both the transport element and the microsensor may be fabricated using micromachining or nanofabrication techniques. In one embodiment, an output of the transport element is coupled to the input of a microsensor. The targeted substance can be retained in a storage vessel for further analysis. The system may be integrated into a handheld device using disposable cartridges for detecting different microorganisms or contaminants.

### RELATED APPLICATION

This application claims priority to U.S. application Ser. No. 60/302,273, filed Jun. 29, 2001, the specification of which is incorporated by reference.

### TECHNICAL FIELD OF THE INVENTION

The present invention relates generally to the field of microorganism and contaminant sensors and, in particular, to a sensor for detecting microorganisms and contaminants in a sample fluid by measuring various dielectric properties of the sample fluid.

### BACKGROUND

Waterborne, pathogenic microorganisms are a significant threat to public health and safety. Microorganisms, such as Cryptosporidium parvum (hereinafter “C. parvum”) thrive in fresh water from which drinking water is drawn. For public health and safety reasons, the detection and removal of such organisms from water and liquid food sources is important.

Animal feces can contaminate water supplies by runoff from agricultural land. In sufficient quantity, the protozoan C. parvum can lead to mild, self-limiting or extremely severe gastroenteritis in humans. Under certain conditions, ingestion of C. parvum can be fatal. One particular instance of C. parvum infection occurred in Milwaukee, Wis. in April 1993 wherein 400,000 people were infected and several deaths resulted.

C. parvum is a coccidian parasitic protozoan that grows within its host which are, typically, humans or farm animals. The protozoan usually enters the environment through the fecal matter of infected domesticated farm animals. It is believed that C. parvum is the only known species, within the genus Cryptosporidium, that is harmful to both humans and livestock. C. parvum is also capable of living in diverse environmental conditions outside the human host as well. When the sporozoite are contained within an oocyst, C. parvum is exceptionally resilient to environmental conditions. The oocyst, typically 4-6 μm in diameter, is the infective stage of the life cycle of C. parvum. The transmissive form of Cryptosporidium parvum is referred to as an oocyst.

FIG. 1 illustrates a microscopic image of three forms of C. parvum oocysts, with sporozoite visible within each oocyst. The upper portion of the image illustrates untreated oocysts, of which it is believed that 97.0% are infective. The middle portion of the image illustrates freeze-thawed oocysts, of which it is believed 1.8% are infective. The lower portion of the image illustrates desiccated oocysts, of which it is believed none are infective.

Oocysts are excreted from infected animals. Within a host, oocysts exist and reproduce in the gastrointestinal tract. Since C. parvum only reproduces within a host, the concentration of microorganisms per volume decreases with distance from the source of contamination. Therefore, the goal of eliminating contamination amounts to identifying and disinfecting the C. parvum host.

The disease Cryptosporidiosis occurs when an animal ingest C. parvum oocysts. Diarrhea and other intestinal maladies are typical with Cryptosporidiosis. Healthy adult human immune systems can defeat Cryptosporidiosis without medical intervention, however, C. parvum infection remains a threat to children, the elderly and those with immune deficiencies, such as acquired immune disease (AIDS). It is believed that there is no treatment for Cryptosporidiosis, thus leaving only supportive therapy. Knowledge of C. parvum concentration and distribution in the drinking water may serve to reduce outbreaks of Cryptosporidiosis.

The United States Environmental Protection Agency (EPA) has published guidelines for controlling potential non-point sources of oocysts. Nevertheless, difficulty in detecting the C. parvum microorganism has precluded any systematic studies to examine the effects of such guidelines. For example, the effects of management practices on transport of soils treated with animal wastes from infected herds remains largely unknown. Similarly, the effects of management practices on water sources also remains unknown. Despite the lack of effectiveness studies, farmers and others may soon be required to employ potentially costly waste management practices. In addition, producers of fresh apple cider may soon be required to pasteurize their product before sale. Costs and burdens associated with new pasteurization and management practices may further escalate consumer prices for agricultural products.

For example, the EPA currently regulates Cryptosporidium concentrations in drinking water through the Interim Enhanced Surface Water Treatment Rule, scheduled to go into effect in December 2001. The rule requires physical removal of at least 99% of Cryptosporidium for filtered surface water systems serving at least 10,000 people. Water systems without filtration must implement a watershed control program to protect the water supply from Cryptosporidium contamination. Municipalities attempting to comply with the rule may encounter compliance problems since most Cryptosporidium detection methods are too slow to prevent outbreaks. Municipalities, and others, recognize the importance of continuous monitoring of source water supplies for C. parvum.

Typically, sorting and isolating a microorganism, or microorganisms, of interest from a fluidic sample, has entailed porous filtration, immunimagnetic separation, flocculation or flow cytometric cell sorting. Porous filtration has been used to recover C. parvum and Giradia (another parasite) from stream water. The EPA has recognized porous filtration, which uses polymeric microfilters, as part of a standardized method for sorting drinking water. Porous filtration enjoys the benefits of low costs and minimal expertise requirements. Filters used with porous filtration include Nuclpore disks and Gelman capsules with typical recovery rates of 39% (standard deviation 13%) and 47% (standard deviation 19%), respectively. In addition to the relatively low recovery rates, porous filtration often includes similar sized microorganisms that later may be mistaken for C. parvum.

Immunimagnetic separation (hereinafter “IMS”) can be used after larger particles and organisms (such as algae and large bacteria) have been removed from a sample. The fluid sample is passed through a column containing magnetic beads coated with antibodies for a specific microorganism. IMS exhibits low recovery rates as well as high variability in the results. Average recovery rates of 75%, with standard deviation of 21%, have been documented. The high variability is believed to be the result of high turbidity of the water sample with high recovery rates associated with low turbidity and low recovery rates associated with high turbidity. According to one study involving samples from sites with different turbidity, IMS exhibited an average recovery rate of 1.82%. A. Zezulak, D. Sharp, P. London, C. Owen. Determination of the Efficacy of Using Immunomagnetic Separation to Remove Cryptosporidium parvum from Various Water Samples versus the Traditional ICR Protozoan Method, in Water Quality Technology Conference Proceedings. 1999, Tampa, Fla.: American Water Works Association. IMS enjoys a low detection limit, namely, just a couple of oocysts, and yet the recovery rate is poor.

However, according to another study, a recovery rate of 95% of the oocysts was achieved using magnet activated cell sorting IMS with C. parvum oocysts using anti-C. parvum antibodies and filtering through a high gradient separation column. M. Q. Deng, K. M. Lam, D. O. Cliver, Immunomagnetic Separation of Cryptosporidium Parvum Oocysts Using MACS MicroBeads and High Gradient Separation Columns. Journal of Microbiological Methods, 1999, 40: p. 11-17. The large differences in recovery rates renders IMS unreliable for sorting procedures. In addition, IMS is recognized as slow and inaccurate for C. parvum sorting requirements.

Flocculation relies on aggregation to increase the particle size and facilitate filtration. Calcium carbonate, aluminum sulphate and ferric sulphate have been used to increase the particle size of C. parvum oocysts. The precipitation process generates small particles that are held in suspension by electrostatic surface charges. The electrostatic surface charges cause clouds of counter-ions to form around the particles, thus giving rise to repulsive forces that prevent aggregation and reduce the effectiveness of later separation processes. Flocculation entails adding coagulants and slow, or low-shear, mixing to promote contact between the coagulant and the particles, and to facilitate sedimentation through flocculation settling. The settled flocculated particles can be collected and examined for C. parvum or other microorganisms. With low turbidity, the highest recovery rate, using aluminum sulphate as the coagulate, is 84.4%. With greater turbidity, lower recovery rates are common, typically in the range of 50-75%. As compared to porous filtration, both IMS and flocculation are more time consuming and costly with marginally improved results.

Flow cytometric cell sorting (FCCS) measures light scatter and fluorescence of particles passing an illuminated zone. Particles, or cells, are pumped sequentially through the illuminated zone at typically 1,000 cells or more per second. The successive scattering and fluorescence signals generated by each passing particle are detected by photo multiplier tubes or photo diodes and correlated into digital data. FCCS can simultaneously sort and detect C. parvum. FCCS is both faster and less labor intensive than the other sorting methods. Recovery efficiency can range from less than 10 to 100%, and in at least one study, IMS was found to be better at isolating Cryptosporidium oocysts than porous filtration, immunimagnetic separation or flocculation. Typically, analysts have found that accurate results are possible only by combining multiple sorting and isolation methods for each particular sample. Such redundancy is wasteful of resources and adds substantially to costs and time delays.

As for detecting a microorganism of interest, in a fluidic sample, typically, methods rely on immunofluorescence assay (hereinafter “IFA”), FCCS and various other molecular detection methods. It is believed that the most widely used method of detecting C. parvum is the IFA technique in which the sample fluid is filtered, eluted with detergent, and centrifuged to concentrate and separate the Cryptosporidium oocysts. Microscopic examination of the fluorescent sample is performed to count the oocysts. One disadvantage of IFA relates to the fact that the IFA test is not species specific. According to one study, IFA tests cross-react 76% of the time with non-C. parvum species. T. K. Graczyk, M. R. Cranfield, R. Fayer, Evaluation of commercial enzyme immunoassay (EIA) and immunofluorescent (IFA) test kits for detection of Cryptosporidium oocysts of species other than Cryptosporidium parvum. American Journal of Tropical Medicine and Hygiene, 1996. 54(3): p. 274-279.

Other problems arise with the use of fluorescent antibody stains. Recent studies show that the IFA method may provide limited information concerning the viability of the protozoa. Earlier studies have shown that the IFA method was not able to distinguish between viable and non-viable oocysts. Id. Viability is a measure of the ability of a microorganism, such as an oocyst, to infect humans or animals. Other methods to determine viability must be done after sorting and counting. IFA also requires manual counting of individual oocysts. Manual counting is both labor intense and prone to human error, especially when counting reaches into the levels of thousands of oocysts. Skilled and experienced microscopists must distinguish between C. parvum oocysts and protozoal and algal species oocysts. Furthermore, current research is inconclusive as to a relationship between viability and infectiousness for humans. One study has shown that as little as 10 oocysts can trigger infection and yet another study has shown that a 50% infectious does (ID50) for humans entailed 132 oocysts. Attempts to automate, or semi-automate, the IFA process have little or no statistical improvement. In addition, IFA is recognized as time consuming with some analysis requiring up to a full day. Such delays, and other problems with IFA, preclude widespread reliance on IFA for the detection of C. parvum oocysts.

FCCS, as described above, can be used to purify and determine the concentration of particles in a sample. FCCS entails analysis of a scattered laser light beam passing through each particle in the suspension and measuring the emitted fluorescent light from each particle. FCCS can also sort cells by measuring light scattering and fluorescent properties of each particle in the sample stream. User-specified light scatter and fluorescent criteria enables grouping of the particles, or cells, by electrically charging each particle and deflecting into an appropriate receptacle.

As compared to IFA, FCCS requires less microscopy time, less operator expertise and enjoys a lower cost per sample. On the other hand, equipment costs for FCCS can be prohibitive, ranging between $115,000 and $200,000. Also, FCCS equipment is not generally portable and thus, complexities associated with transporting a fluid sample can adversely impact the analysis. In addition, FCCS requires clean water samples. Thus, liquid foods and soiled water cannot be processed using FCCS without filtering the sample. Filtering, however, generally causes a loss of oocysts. The best results are obtained with the least turbid water samples. Another shortcoming of FCCS is an inability to distinguish between autofluorescent algae and mineral particles in water samples. The sensitivity of FCCS is poor when fewer than 100 oocysts are present in the sample.

Other methods for molecular detection of C. parvum protozoa include polymerase chain reaction (PCR) based detection methods. For example, one such assay, known as IMS-PCR combines an IMS sorting of oocysts with a specific amplification step. Unlike IFA methods, PCR is species specific. Thus, using PCR, it is possible to separately detect C. parvum from other Cryptosporidium species. However, PCR does not provide a dependable measurement of the amount of oocysts, and consequently, most PCR assays are referred to as “presence-absence tests.”

The sensitivity of PCR, particularly with regard to soil studies, is poor at low oocysts concentrations. Specific PCR primers with high sensitivity have demonstrated an ability to detect as few as four oocysts in a sample fluid having an approximate volume of 1 mL following a concentration process performed on a much larger volume of fluid. One disadvantage of PCR is it takes several hours to process a sample, and thus, results are not immediately available. Also, PCR requires operator expertise to produce precise results.

Other molecular detection methods include reverse transcription or ribosomal RNA oligonucleotode probes. Such methods, although they can be performed in situ, and can detect the presence of C. parvum oocysts, are incapable of quantifying or determining the viability of particular oocysts. Another method uses a combination of flow cytometer sorting, amplification with molecular techniques and visual detection using monoclonal fluorescent antibodies. This method can detect 10 oocysts per liter in a period of about 5 hours and requires little operator expertise. Nevertheless, this method is imprecise because it is qualitative and relies on visual techniques. Other methods also involve combinations of existing techniques, such as fluorescent staining and PCR or IMS and PCR. Vital dye and excystation assay methods are also available but they lack efficiency and accurate quantification of oocysts.

For the reasons stated above, and for other reasons stated below which will become apparent to those skilled in the art upon reading and understanding the present specification, there is a need in the art for a system and method that allows for rapid and accurate monitoring of quality and food for undesirable, and unsafe, contamination.

### SUMMARY

The above mentioned problems concerning contamination of fluids are addressed by the present invention and will be understood by reading and studying the following specification. A system and method are described for detecting microorganisms in a fluid.

By way of overview, the system includes a capillary element for isolating, and thus concentrating, a preselected component of a fluid sample. The capillary element, or flow cell, may be fabricated as a planar element made of silicone elastomer using micromachining or nanofabrication techniques. As a planar element, the input and output ports are along a first and second edge. An interior surface of the capillary element may be coated with an immobilized binding partner for a microorganism, or microorganisms, of interest. The microorganism may be a pathogenic microorganism. For example, the immobilized binding partner may be an antibody or fragment thereof that binds to a particular analyte. Preferably, the binding partner binds specifically to said microorganism. In one embodiment, the antibody binds to C. parvum. The capillary element thus employs immunocapture to select and concentrate the microorganism of interest from the sample.

The system includes a microsensor element designed to detect the microorganism of interest. The microsensor, or microchip, employs an electric field passing between metal electrodes within a nanofabricated channel to determine a dielectric property of the microorganism in the channel. The numerosity and viability of microorganisms passing the channel are detectable. The dielectric property may include a dielectric constant, a dielectric loss, a dielectric breakdown voltage, a dielectric strength, or a dielectric absorption of the microorganism. In the case of C. parvum bacterium, the viability can be determined by measuring the dielectric properties of the walls of the oocyst.

One embodiment provides that the microorganisms of interest captured in the capillary element are eluted and passed to the microsensor element for detection and counting. The system may also include a storage vessel for retaining the gathered microorganism, or microorganisms, after detection, to enable further analysis. The system may be integrated into a handheld battery operated device using disposable cartridges. A cartridge may be selected based on the type of sample fluid being tested.

As used herein, the term microorganism includes intact functional microorganisms such as bacteria, fungi, yeast, viruses, protozoa, amoeba, spores, and the like, as well as fragments or subunits thereof, including buds, spores, oocysts, and the like.

### BRIEF DESCRIPTION OF THE DRAWINGS

FIG. 1 illustrates a microscopic image of three forms of C. parvum oocysts, with sporozoite visible within each oocyst.

FIG. 2 illustrates a design for a microorganism detection system according to one embodiment of the present system.

FIG. 3A illustrates an isolator flow cell pattern

FIG. 3B illustrates an isolator flow cell pattern.

FIG. 4 graphically illustrates a schematic of a flow cell configuration according to one embodiment of the present system.

FIG. 5 graphically illustrates a first photomask for a nanofabricated detector.

FIG. 6 graphically illustrates a second photomask for a nanofabricated detector.

FIGS. 7A, 7B, 7C, 7D, 7E, 7F, 7G and 7H illustrate a method of fabricating a sensor according to one embodiment of the present system.

FIG. 8 illustrates an embodiment of a sensor wired for determining a dielectric property of a microorganism.

FIG. 9 illustrates a first view of a scanning electron image of a channel.

FIGS. 10A and 10B illustrate views of a scanning electron image of a channel.

FIG. 11 illustrates a channel.

FIG. 12 illustrates a channel with gold electrodes.

FIGS. 13A, 13B, 13C and 13D graphically illustrate performance for one embodiment of the present system.

FIG. 14 illustrates a perspective view of a handheld embodiment of the present system.

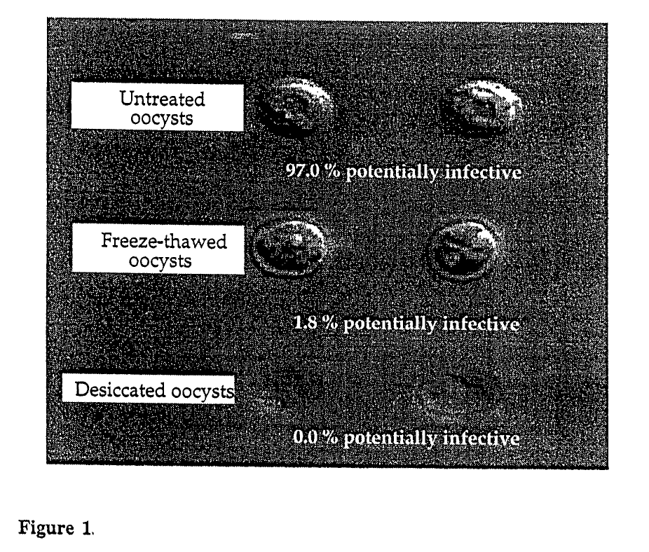

### DETAILED DESCRIPTION

The following detailed description refers to the accompanying drawings which form a part of the specification. The drawings show, and the detailed description describes, by way of illustration specific illustrative embodiments in which the invention may be practiced. These embodiments are described in sufficient detail to enable those skilled in the art to practice the invention. Other embodiments may be used and logical, mechanical, electrical and chemical changes may be made without departing from the scope of the present invention. The following detailed description is, therefore, not to be taken in a limiting sense.

The detection system described herein isolates and detects microorganisms in liquid samples, particularly, pathogenic microorganisms. The system also can indicate the viability of intact, detected microorganisms, such as oocysts. A portion of the system is sensitive to the dielectric properties of individual microorganisms. The system may also include a storage vessel for retaining the microorganisms after detection and, optionally, determination of viability.

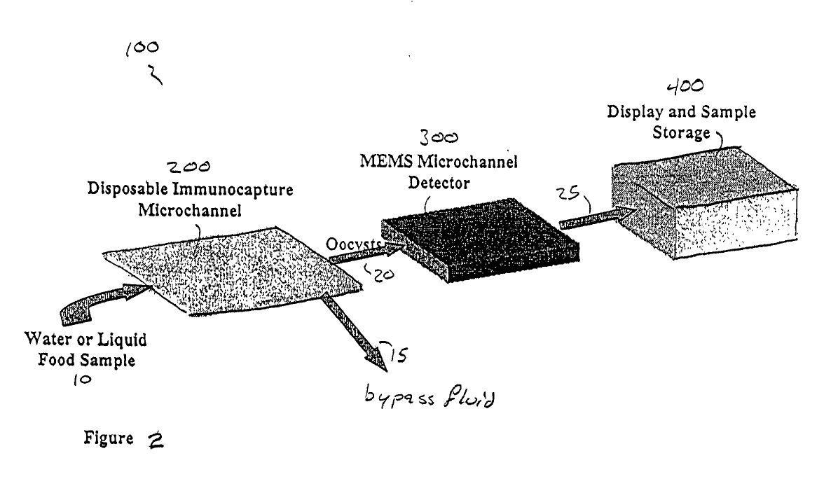

FIG. 2 illustrates a design for a microorganism detection system 100, according to one embodiment of the present system, having both microchannel (or isolator) 200 and detector 300. The output from microchannel 200 includes bypass fluid 15 and microorganisms 20. Bypass fluid 15 represents the fluid sample after the microorganisms 20 are filtered out. Microorganisms 20 are routed to the input of detector 300. The output from detector 300 includes detected microorganisms, marked 25 in the figure, and is routed to element 400. Element 400 displays the analysis results. In the embodiment shown, element 400 also provides storage for detected microorganisms. Retained microorganisms may be later analyzed using other equipment or techniques.

It will be appreciated that the microchannel 200 may operate independent of detector 300. Also, detector 300 may operate independent of microchannel 200. In other words, microchannel 200 may provide isolated microorganisms to other detectors or sensors and detector 300 may be used with other isolation or purification means.

Isolator

Isolator 200 employs a capillary system for isolating a particular microorganism from a sample of fluid. The sample may or may not include a particular microorganism.

Isolator 200 includes a system of pathways that performs affinity purification of the sample fluid. The pathways of the isolator operate akin to porous filtration media. for transporting and filtering the sample fluid. The sample fluid may be injected into the isolator and transported through the flow paths by capillary action.

A binding partner for the microorganism, or subunit thereof, may be immobilized on the interior surface of the isolator. Isolator 200, along with the binding partner, performs affinity purification. Affinity purification relies on the ability of the antibody to bind to and to isolate the microorganism. Affinity purification is effective not only for hapten-specific, peptide-specific, but antigen-specific antibody populations as well. Isolator 200 may filter the sample fluid using an immobilized monoclonal antibody, a polyclonal antibody, or a binding fragment thereof. Isolator 200 may also utilize an APTase, or an RNA APTase of a microorganism of interest as a binding partner. Isolator 200 may include a binding partner that preferentially attaches to a predetermined microbe.

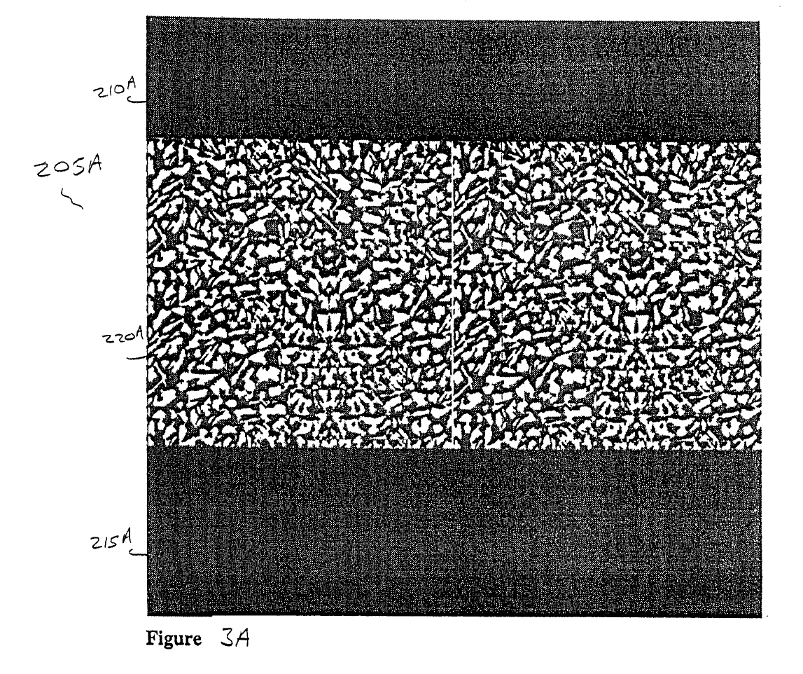

Isolator 200 may include a pattern of pathways designed to simulate a porous media filter. In one embodiment, the pattern may appear as a labyrinth of intricate passageways. Isolator 200 may be fabricated by a mold process or it may be fabricated using photolithography procedures including photoresist and etchants. FIG. 3A illustrates image 205A of a pattern of pathways 220A for use in fabricating isolator 200 by molding. Image 205A illustrates a negative image of a pattern to be etched into a silicon wafer. The etched wafer is subsequently used as a mold for casting the silicone elastomer isolator. In FIG. 3A, the white regions represent areas where a silicon wafer is etched and the dark regions represent areas not etched. Consequently, when the silicon wafer is later used as a mold, the areas appearing white, in FIG. 3A, will result in walls of isolator 200 and the areas appearing dark will result in fluid pathways.

In FIG. 3A, rectangular region 210A corresponds to the inlet to the isolator 200 and region 215A corresponds to the outlet of isolator 200. A large number of pathways 220B connect inlet 210A to outlet 220A. Pathways 220A presents a large surface area with which the sample fluid traverses. The large surface area increases the likelihood of a single organism of interest contacting and binding to a binding partner.

FIG. 3B illustrates an alternative pattern of a pathway for isolator 200. Image 205B illustrates a negative image of a pattern to be etched into a silicon wafer. As explained with regard to FIG. 3A, the white regions represent areas where a silicon wafer is etched and the dark regions represent areas not etched and thus, the areas appearing white will result in walls and dark areas will result in fluid pathways. In FIG. 3B, inlet port 210B is coupled to a pathway 220B. Pathway 220B is arranged in a serpentine pattern and includes a plurality of angled ridges 222. Ridges 222 provide an increased surface area to enhance the attachment of an immobilized binding partner.

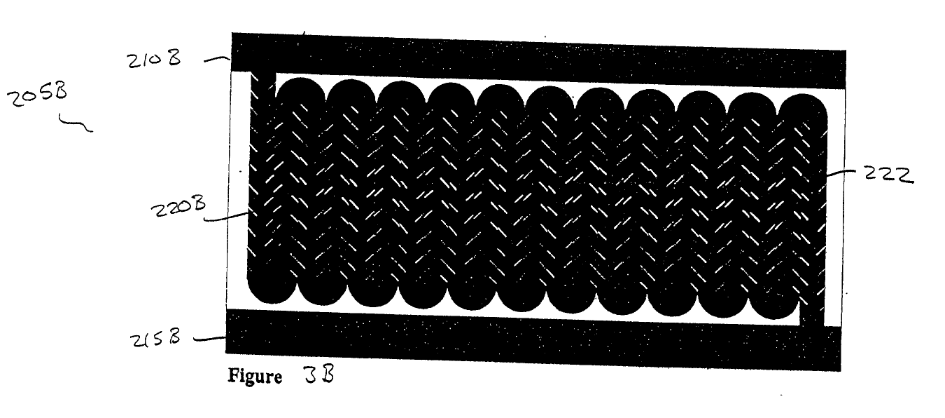

FIG. 4 graphically illustrates a schematic of a flow cell configuration for use with a molded isolator element, according to one embodiment of the present system. In the figure, system 250 includes a work surface 255 having a vacuum connection 245, a fluid input 231 and a fluid output 236. Isolator 200 includes an input port 211 and an output port 216 separated by a network, or system, of pathways 221. Input port 211 is coupled to fluid input 231 and output port 216 is coupled to fluid output 236. In the embodiment shown, isolator 200 is fabricated of molded silicone elastomer. Useful silicone elastomers are disposable, biodegradable, and inexpensive. Antibodies, antibody fragments or subunits thereof, and other functional proteins, can bond readily to silicone elastomer. Isolator 200 is bonded to surface 225. The bond between surface 225 and isolator 200 is leakproof for the sample fluid of interest. A vacuum is applied to connection 245 and conveyed by line 240 to surface 225. The vacuum provides an external force to facilitate achievement of a leakproof coupling between surface 225 and isolator 200.

Fabrication of isolator 200 may include a photolithography process or other semiconductor processing techniques for the fabrication of microcapillary channels of isolator 200. Silicon wafer processing is described in Madou, M.. Fundamentals of Microfabrication, 1997, Boca Raton, Fla.: CRC Press, and an adaptation of such methods is used for the fabrication of isolator 200 using a 3″ silicon wafer. The following is a detailed description for preparing a mold using a silicon wafer, according to one embodiment:

A pattern was prepared using SYMBAD computer aided design software from Cadence Design Systems, Inc. Pattern 205A of FIG. 3A was copied onto a photomask master plate with a finished size of 68 mm by 68 mm. The photomask was a 5″ glass plate with the pattern consisting of a chrome layer above the glass surface.

The pattern on the photomask was transferred to silicon wafers through ultraviolet light exposure of an intermediate photoresist layer using a 5× g-line stepper. The layer of photoresist, here, Olin-Hunt HiPR 6512, covering the top surface of the wafer was spun on at 3,150 rpm for a period of 40 seconds. The wafer was then pre-baked by placing on a 90° C. hotplate for a period of 60 seconds. The resulting photoresist layer was approximately 1.6 μm in thickness. The wafer, with photoresist, was then aligned with the photomask and exposed to UV light at exposure settings sufficient to properly expose the photoresist layer. The wafer was then post-baked by placing on a 90° C. hotplate for a period of 60 seconds. Following cooling from the post-bake procedure, the wafer was then placed in developer solution (MF CD 26, Shipley Microelectronics) for 1 minute, thus allowing the pattern 205A to appear in the resist film by washing away the exposed photoresist. The wafer was then rinsed with deionized water and air dried.

The pattern was then transferred to the underlying silicon substrate by means of a dry chlorine Cl₂ etching process. The dry Cl₂ etching process used an ion coupled plasma (“ICP”) etcher running a Robert Bosch GmbH process. The Bosch process etches silicon at a rate of approximately 2 μm per minute. Several silicon wafers were prepared having etch depths of 10 μm, 20 μm and 40 μm. The remaining photoresist was wet stripped from the silicon wafer using photoresist stripper 1165 Remover (Shipley, Whitehall, Pa.). The wafer was then rinsed with deionized water and air dried.

To facilitate the later release of the mold, the several wafers were placed in a 100% atmosphere of N₂ and then coated, by vapor deposition, with 1H, 1H, 2H, 2H-perflourodecyltrichlorosilane (Lancaster, Lancaster, Pa.) before making the silicone elastomer isolator. The wafer is placed into a box that had been vacuum pumped and purged with N₂, with a 10 mL vial containing a few milliliters of the liquid form of the compound. Both the wafer and the vial were placed in a petri dish and covered with parafilm to keep the vapor from falling outside the dish. The wafers were exposed for a period of between 2 and 3 hours, resulting in a coating having a thickness of about 100 Å. Following vapor deposition, the wafer was used as a mold from which silicone elastomer isolators 200 were made.

Using the silicon wafer as a mold, a silicone elastomer isolator 200 was prepared using the following method:

Room temperature vulcanizing silicone elastomer, one example of which is known commercially as RTV 615 (General Electric, Waterford, N.Y.), was used for isolator 200. RTV 615 is a two-part mixture of vinylmethylpolysiloxane and polydimethylhydrogensiloxane and is mixed together at a mass ratio of 10:1. The mixture was placed in an evacuated flask for a period of 30 minutes and stirred with a magnetic stir bar to displace dissolved oxygen and other gases. The RTV 615 was then poured over the silicon wafer and cured in a 65° C. oven for a period of approximately one hour. Curing may also be done at 60° C. for 2 hours or at room temperature for a longer period of time. Curing time can be reduced using a higher temperature, and in one embodiment, curing temperature of up to 150° C. may be used. At elevated temperatures, the elastomer will start releasing toxic gases.

After cooling to room temperature, the patterned silicone elastomer was diced using a sharp knife and removed from the silicon wafer by peeling. The elastomer was hermetically bonded to glass to form the capillary column. The diced silicone elastomer segments were then used as part of isolator 200.

Fluid flow resistance and structural strength are determined, in part, by the overall thickness of the elastomer capillary column. A column with a thickness of approximately 1 mm yields satisfactory results. A column that is thinner than 1 mm may lack sufficient tensile strength, and thus, not provide adequate structural support. A column thicker than 1 mm may be used. C. parvum oocysts can be recovered from the column using a concentrated solution using a citrate-phosphate buffer at a pH of approximately 3.5.

One embodiment of the present subject matter includes additional structural strength derived from a coating of reinforcing material applied to the column. The reinforcing material may include hot melted spin glass or other material. Other structures or materials may also be used to increase the rigidity.

In one embodiment, the column operates in an electromagnetic field. The electromagnetic field augments dissociation provided by the buffer solution. The field may be generated by an electrode array positioned below the column or by electrodes placed adjacent to the column. Other means of augmenting dissociation may also be utilized, including optical or thermal means.

Various means may be utilized to immobilize the binding partner to the silicone elastomer. For example, in one embodiment, suitable conjugate pairs may be utilized. Other means of immobilizing the binding partner are also contemplated.

Isolator 200 may operate to isolate microorganisms using a continuous flow of sample fluid comprising said microorganism. Isolator 200 may also isolate microorganisms using a static sample fluid supply.

Detector

Detector 300 includes a narrow passageway leading to an orifice for capturing and retaining the microorganism. An electromagnetic field in the orifice enables measurement of dielectric properties indicative of the presence of a microorganism as well as the viability of any detected microorganism. Fabrication of detector 300 may entail photolithography, bulk silicon micromachining, application of thin films, etching of channels in silicon and application of a metal coating to side walls of a channel. In one embodiment, the channel is 10 μm deep and 4-6 μm wide. Fabrication of one embodiment of detector 300 is as follows:

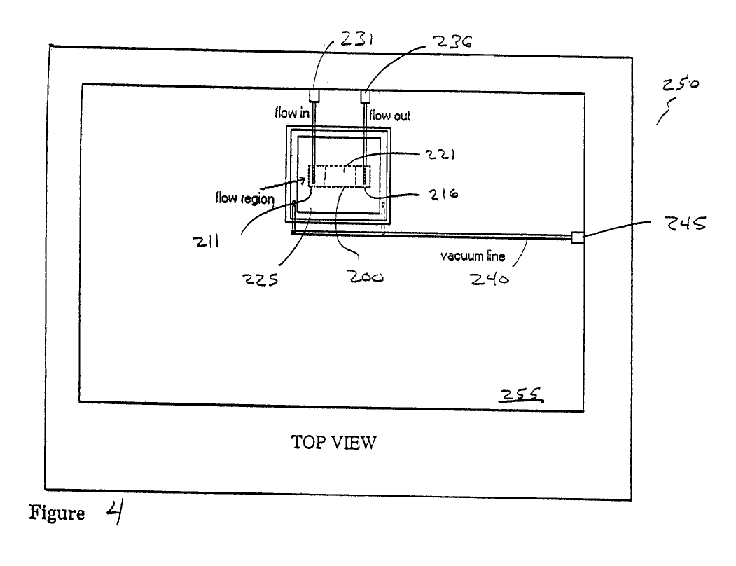

A pattern for detector 300 was designed using SYMBAD and transferred to chrome on a 5″ glass plate. The dimensions of the pattern on the glass plate was 63.01 mm by 63.265 mm. A pattern for the first layer is illustrated in FIG. 5 and marked 305. Visible in pattern 305 is an inlet region 310, outlet region 340 and three channels. The first channel has inlet side 315, orifice 317 and outlet side 319. The second channel has inlet side 325, orifice 327 and outlet side 329. The third channel has inlet side 335, orifice 337 and outlet side 339. Orifices 317, 327 and 337 have cross sectional dimensions of 4 μm, 6 μm and 8 μm, respectively.

Shipley 1813 photoresist was spun onto 3″ silicon wafers at 2500 rpm for a period of 30 seconds, resulting in a photoresist thickness of approximately 1.7 μm. The silicon wafers were then baked on a hotplate at 115° C. for a period of 60 seconds before exposure for solvent removal and stress annealing. The wafers were then exposed to UV light in a 5× g-line stepper with the following inputs: exposure time of 1 second and a focus of 251. The wafers were then post-baked by placing on a 90° C. hotplate for a period of 60 seconds. After cooling for approximately one minute, the wafers were then placed in MF CD 26 developer solution for a period of one minute. The wafers were then rinsed with deionized water for 15 seconds and air dried. Using the patterned photoresist layer as a protective mask, the wafers were then placed in the ICP etcher for a period of 5 minutes, in accordance with the Bosch “O-trench” batch file process. This procedure etches silicon at a rate of approximately 2 μm per minute, thus producing channels having a depth of approximately 10 μm. The photoresist was then stripped off the wafers using an oxygen plasma process for a period of approximately 11 minutes.

A layer of silicon dioxide (SiO₂) was then deposited onto the wafers by plasma enhanced chemical vapor deposition (PECVD). The oxide deposition rate was 32.5 nm per minute and the exposure had a duration of 15 minutes, thus yielding a layer having a thickness of approximately 500 nm.

Depositioning and patterning of the metal layers followed. Two conformal layers of metal were applied by sputtering directly over the oxide layer. The first layer was a chrome layer having an approximate thickness of 200 nm. The second layer was a gold layer having an approximate thickness of 350 nm. Patterning of the gold contacts, or electrodes, occurred after depositing the metal layers. The detailed description of the depositioning and patterning of the metal layers is as follows:

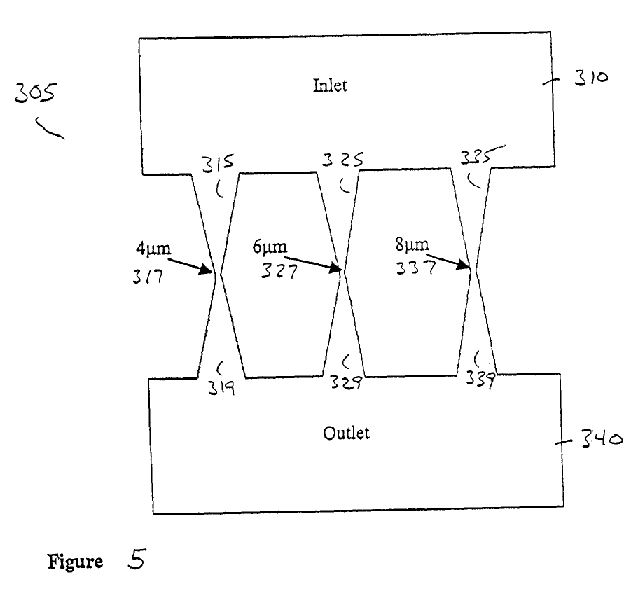

An 18 μm layer of high density photoresist was spun onto the wafers at 5,000 rpm for a period of 30 seconds. In the procedure described herein, the photoresist used is commercially known as AZ 4903 and has a specific gravity of 1.09±0.01. The wafers were then pre-baked at 115° C. for a duration of 90 seconds. Next, the wafers were incubated for 1.5 hours. The photoresist then was exposed in the 5× g-line stepper, using second mask layer 350 illustrated in FIG. 6. Pattern 350 includes gold metal contacts, or electrodes, 317A and 317B positioned at orifice 317, contacts 327A and 327B positioned at orifice 327, and contacts 337A and 337B positioned at orifice 337. The exposure had a duration of 2.5 seconds and a focus of 287. The wafer was then developed in a solution of MF CD 26 developer for a period of one minute. The wafer was then rinsed in deionized water for approximately 15 seconds and air dried.

The metal layer was etched using Transene Gold etchant, having an etch rate of 28 Å per second. A volume of 25 mL of etchant was placed in a beaker and the wafers were immersed for a period of approximately 125 seconds. The etchant removed both the gold and the chrome in areas not protected by the photoresist. Following etching, the photoresist was stripped in an oxygen plasma process for approximately 10 minutes.

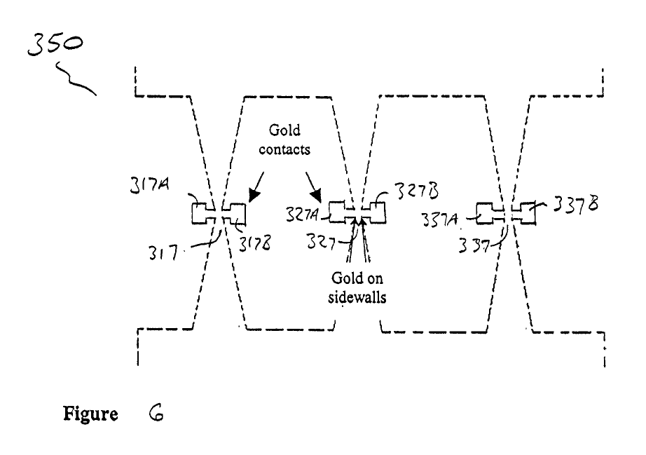

This procedure is outlined in FIGS. 7A through 7H. An edge view of silicon wafer 360 appears in each of the figures. FIG. 7B illustrates the addition of photoresist 365 layer to wafer 360. FIG. 7C illustrates the results following etching of a channel, herein denoted as 370. It will be appreciated that channel 370 is one embodiment of a channel and in the masks illustrated in FIGS. 5 and 6, three such channels were depicted. In FIG. 7D, silicon dioxide layer 375 is applied to wafer 360 and channel 370. In FIG. 7E, gold layer 380 is applied to silicon dioxide layer 375. In FIG. 7F, photoresist 385 is applied to gold layer 380. In FIG. 7G, gold layer 380 has been etched. In FIG. 7H, photoresist 385 is removed, leaving gold contacts in the orifice portion of channel 370.

In one embodiment, detector 300 is configured to capture and retain microorganisms and allow the passage of the sample fluid. The sample fluid may include an aqueous fluid such as water, liquid lipids or organic solvents. Other examples of sample fluids include milk, juice (including fruit juice or vegetable juice), cider (including apple cider) or other liquid foods. Body fluids may also be used with the present system. For example, blood, blood products (including plasma), urine, secretions, or excretions may be used. In addition, atmospheric gases or vapors may be utilized with the present system. Dielectric properties of retained microorganisms are determined using electrical measurement techniques, thus indicating the type and viability of microorganisms.

Prototype

Continuing with the example described above, dies are cut from silicon wafer 360 and the contacts were electrically coupled to a chip socket. In one embodiment, the die is bonded with double-sided tape to a 12-pin chip socket. The die and socket may be wire bonded using a two-step bonder such as that commercially known as a Westbond 7400A Ultrasonic Wire Bonder. The socket was placed onto a circuit board for electronic detection of microorganisms such as oocysts.

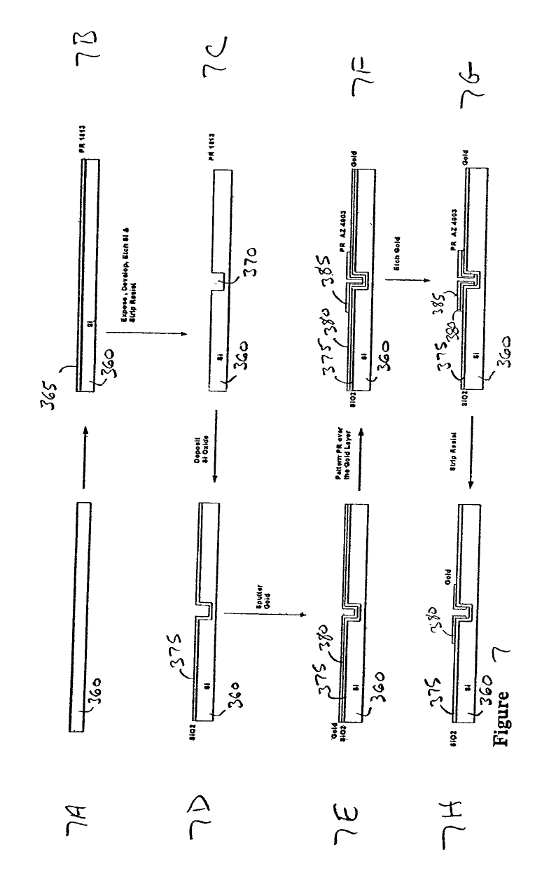

The socket was placed onto breadboard assembly 515 and isolated in aluminum housing 500 illustrated in FIG. 8. Breadboard assembly 515 includes detector 300. Electrical connections to detector 300 are via connector 505 and 510, each mounted to housing 500. Lid 502 is illustrated in the figure in a removed position relative to housing 500.

Electrode 317A is coupled to connector 505. Connector 505 is coupled to a waveform generator via an electrical cable. Electrode 317B is coupled to connector 510. Connector 510 is coupled to a signal analyzer. The test setup herein described can be modeled by a simple resistive-capacitative circuit.

Performance—Isolator

Using the prototype embodiment previously described, the performance was determined using four different fluid samples and one sample consisting of air. A 100 μL aliquot of each sample was placed, by injection, into the channel of detector 300. With the sample fluids in the channel, a 1.0 dBm burst signal at a frequency of 10 kHz was applied to connector 505 using a signal generator (Agilent Wave Generator, Agilent, Palo Alto, Calif.). The burst modulation settings were 1000 Hz, 0.0° phase, and 100 Hz. Connector 510 was coupled to a signal analyzer (Agilent Vector Analyzer, Agilent, Palo Alto, Calif.) for receiving the filtered signal through the channel of detector 300.

The four sample fluids analyzed were (1) no sample (air); (2) deionized water; (3) serum only from a C. parvum vial; (4) serum with live C. parvum; (5) desiccated C. parvum. In one embodiment, the C. parvum was obtained through the National Institute of Health (NIH) AIDS Research and Reference Reagent Program and the bath was prepared using C. parvum in a 2.5% potassium dichromate solution which was further diluted in a phosphorus buffered saline solution of 0.04M K₂HPO₄, 0.01M NaH₂PO₄.H₂O, and 0.124M NaCl. Other concentrations or solutions may also be used.

The silicone elastomer of isolator 200 bonds hermetically with glass, PLEXIGLAS™ (registered by Rohm & Haas Company, Philadelphia, Pa.) silicon, and also silicone as a result of the charge polarization of silica compounds. Such materials may serve as the sealing surface with isolator 200.

The present system may be operated with a static fluid source or with a dynamic fluid source flowing at a predetermined rate. Low fluid flow rates, that is, under 0.2 mL per hour, may be used with isolator 200 in ambient conditions. Moderate, or high (greater than 1.0 mL per hour), flow rates may be used with isolator 200 when using a vacuum to maintain the bond between isolator 200 and the sealing surface. Substantially higher fluid flow rates may require application of a greater vacuum. At sufficiently high flow rates, increasing vacuum may cut off fluid flow, thus suspending operation of system 100. Increasing the depth of the channel in isolator 200 has facilitated a greater flow rate. For example, increasing the channel size from 20 μm to 40 μm enabled an increased flow rate. In one embodiment, the highest controllable flow rate was approximately 0.8 mL per hour when operating under ambient conditions. Other flow rates are also possible, depending, in part, on the design of the channel. With higher sample fluid flow rates, the present system is well suited for real-time testing of fluids.

Performance—Detector

Detector 300 described above enabled easy access to the input and output ports of the channel. Each channel included a narrow passage (orifice) to capture microorganisms, such as oocysts, for initial characterization measurements. The orifice allowed passage of fluid but not passage of the microorganism, i.e., that oocyst. As noted previously, one embodiment employed a series of three channels, each having a different cross sectional dimension. The different sizes facilitated determination of a preferred size for microorganism characterization.

Each channel was fabricated with a depth of between 5 μm and 10 μm. This dimension enabled each oocyst to flow through the channel in single file, or sequential, manner. Individual oocyst flow through each channel allowed accurate characterization of dielectric properties of each oocyst. In the case at hand, the microorganism under investigation was the Cryptosporidium parvum oocyst. Sporozoite wall and oocyst wall have ionic (or electric potentials allowing individual detection). Passage of an oocyst through the field created by the metal electrodes at the orifice of the channel causes a change in the dielectric property of the space between the electrodes. This change in dielectric property causes a detectable change in the output signal, thus indicating the presence of an oocyst.

After fabrication of the channels in silicon, a layer of silicon oxide was applied over the wafer. The oxide layer serves as an electrical insulator and separates the silicon and metal layers. The thickness of the silicon oxide layer may vary but should not be a thickness of more than 1 μm since, preferably, the smallest channel width is 4 μm. The silicon oxide layer varied in thickness between 400 nm and 600 nm and was applied by deposition at a rate of 32.5 nm per minute.

A passing object can be detected by a detecting changes in the properties of the electromagnetic field. The electrodes on the orifice provide the electromagnetic field. In one embodiment, the electrodes appear electrically as two parallel plates of a capacitor. The electrodes may be fabricated of any metal, such as aluminum, silver, cobalt as well as gold (previously described). Semiconductor processes favor the use of aluminum however, rapid oxidation remains a problem with changes in pH. Since sample fluids may have a wide variety of pH values, it may be more desirable to select a metal having more stability with changes in pH. Gold (Au) is one such choice. Gold is not prone to oxidation and it offers good physical, chemical and electrical properties. Gold, however, does not readily bond to silicon dioxide. In the embodiment described above, chrome was used as an adhesion layer between the gold electrode and the silicon dioxide layer.

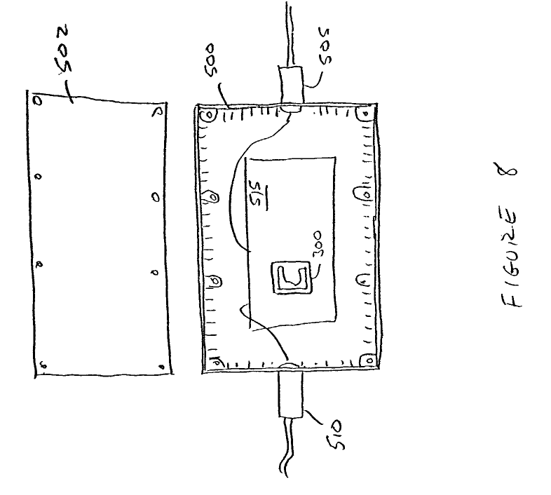

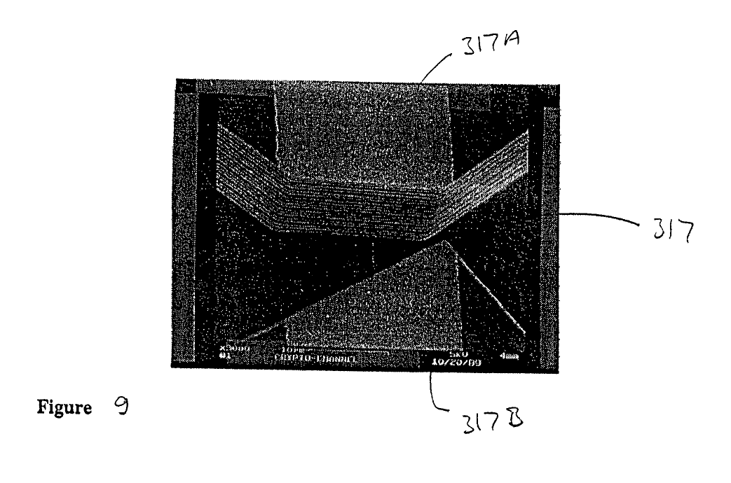

The metal electrode layer may be applied by evaporating or by sputtering. Other methods may also be used. Evaporation produces a non-conformal layer of metal that is evaporated from a crucible and onto the wafer. Sputtering entails firing argon gas atoms at a metal target, thus releasing metal atoms toward the wafer. Either method may be used for the present subject matter, however it appears that sputtering is preferable since this process yielded good metal coverage of the side wall surfaces of the channel. FIG. 9 illustrates metalization of an orifice using evaporation and FIGS. 10A and 10B illustrate metalization by sputtering.

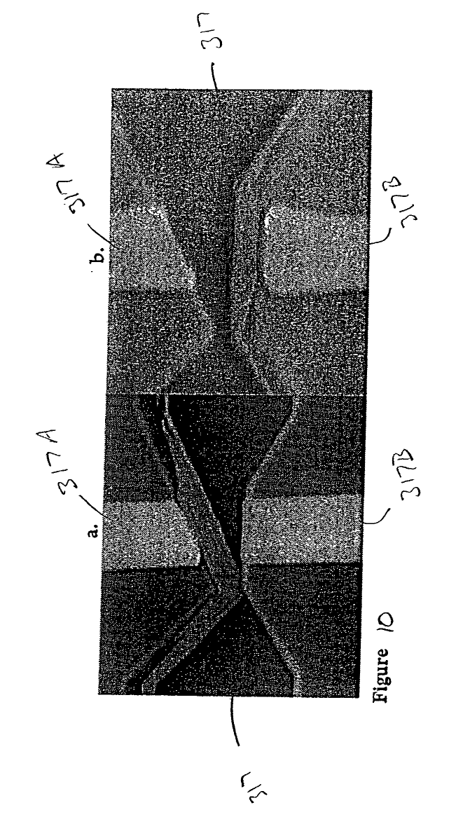

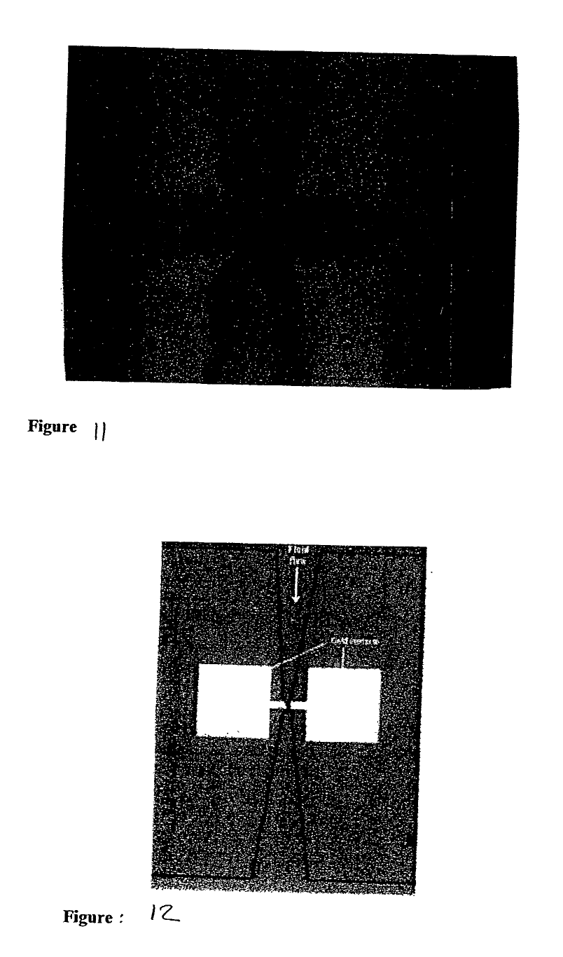

During the fabrication of detector 300, etching of the gold metalization layer proceeds for a predetermined period of time. Proper selection and application of a suitable photoresist layer affects the metal etching process. In one embodiment, Shipley 1813 was used as a photoresist when etching the first layer channels, as illustrated in FIG. 5. Shipley 1813 provides well-defined channels and adequately protected the silicon wafer. However, an improved photoresist is preferred for etching the second layer of the channel. Sidewall profiles showed deterioration at the bottom of the narrow regions when Shipley 1813 was used. In particular, it was noted that the exposed and developed resist was bridged between the two side walls of the channel, as shown in FIG. 11. Nevertheless, the resist profile was satisfactory, and thus, changes were made only to the steps subsequent to the pre-exposure bake. Focus, exposure time in the stepper, and development time did not appear to affect the channel profile. It appears that the channel sidewall profile is determined, at least in part, by the density of the photoresist. The sidewall profile appears to be better with increasing density of photoresist, as shown in FIG. 12. It is noted that Shipley 1813 photoresist was unsatisfactory since it did not suitably coat the sidewalls of the channel with a uniformly thick layer. A denser photoresist, commercially known as AZ 4903, seems to satisfactorily coat the sidewall and thus, define the sidewall metal layer.

An incubation period of 45 minutes was used with the AZ 4903 photoresist, thus allowing for stress annealing and solvent removal. The silicon wafers were removed from sources of white light for a period of between 1.5 and 2 hours, preferably no more than 4 hours, due to degradation of the photoresist.

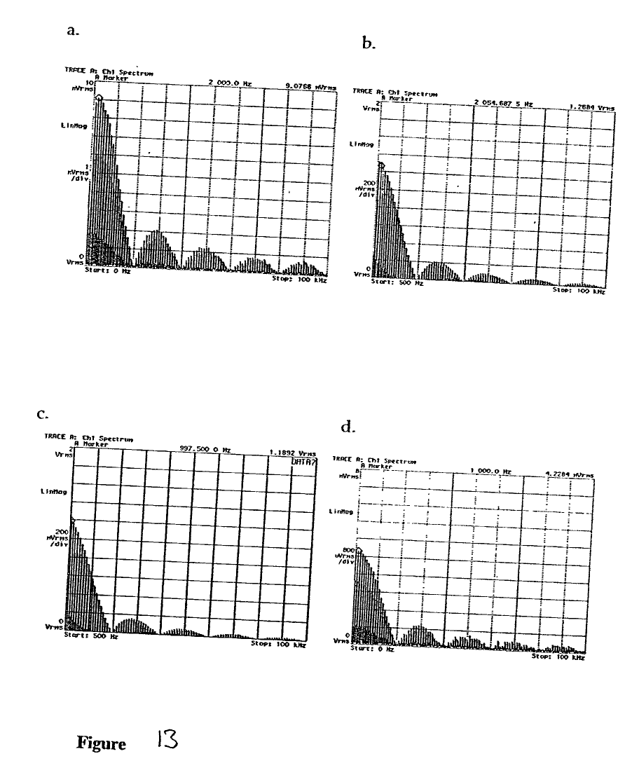

Analysis of the input and output signals using the signal generator and the signal analyzer are illustrated in FIGS. 13A, 13B, 13C and 13D. In each of the figures, the abscissa represents a frequency range, in Hertz, and the ordinate represents a voltage level. The lower frequency portion of the spectrum was used since the high energy of higher frequencies were likely to damage the detector.

In FIG. 13A, for example, no sample fluid was introduced, and thus, the detector operated with an air dielectric. The voltage scale in FIG. 13A is calibrated in mV RMS and appears to have a peak amplitude of approximately 9 mV RMS at a frequency of 2 kHz. In FIG. 13B, the sample fluid included serum only from a C. parvum vial. The voltage scale in FIG. 13B is calibrated in V RMS and appears to exhibit a peak amplitude of approximately 1.2 VRMS at a frequency of 2.054 kHz. In FIG. 3C, the sample fluid includes serum with live C. parvum. The voltage scale in FIG. 3C is calibrated in V RMS and appears to exhibit a peak amplitude of approximately 1.19 V RMS at a frequency of 1000 Hz. In FIG. 13D, the sample fluid included desiccated C. parvum in a 2.5% potassium dichromate solution which was further diluted in a phosphorus buffered saline solution of 0.04M K₂HPO₄, 0.01M NaH₂PO₄•H₂O, and 0.124M NaCl, as previously described. Other concentrations or solutions may also be used. The voltage scale in FIG. 13D is calibrated in mV RMS and appears to exhibit a peak amplitude of approximately 4.23 mV RMS at a frequency of 1000 Hz. It will be noted that variations in the dielectric material (that is, the material between the electrodes) results in variations in the voltage as well as the peak frequency. Differences between viable and non-viable oocysts, are thus detectable.

In one embodiment, the sample fluid is modeled as an unknown black box circuit element. An equivalent circuit for the black box circuit element can be identified by applying an input signal and monitoring the output signal. An unknown microorganism in the sample has, for example, a measurable capacitance and conductance. In one embodiment, the applied input signal includes a waveform or a pulse train. In one embodiment, the pulse train includes a signal which varies between a negative and positive value. In one embodiment, the pulse train includes a signal which varies between a first value and a second value. The first value may be a negative, zero, or positive value, and in general, the second value is greater than the first value. In one embodiment, the pulse train includes a square wave signal. The dwell time, or duty cycle, of the pulse train may be user-selectable. Analysis of the output signal may be performed in the frequency domain or time domain. The applied signal level can be user selectable. In one embodiment, the applied signal is sufficiently low to preserve the cellular integrity of the sample. If the signal level is too high, cell integrity can be degraded by electrolysis within the sample. Electrolysis may cause outgassing and damage to the cell walls. In various embodiments, the pulse train includes a sinusoidal waveform, a single pulse or a square wave.

Alternative Embodiments

Variations in the fluid pathways of isolator 200 may facilitate higher fluid flow rates. Variations also may provide a greater wall surface area, thus facilitating immobilization of a greater amount of binding partner, thus, improving the rate of filtration through isolator 200.

Suitable selection of a fluid pathway pattern may also enhance the retention of captured microorganisms, even at elevated fluid flow rates. One embodiment provides that rather than using Plexiglass as a sealing surface, a second layer of silicone elastomer may be provided. The second layer of silicone elastomer may include a second fluid flow pathways, thus further increasing the surface area for microorganism binding. Such a solution, that is, using two silicone elastomer members, may enable high fluid flow rates without the need for application of an external vacuum.

Contamination on the wall surfaces of isolator 200 may also degrade the bonding of microorganisms to the immobilized binding partners. Elimination of the contaminants is believed to promote greater efficiency of affinity purification. The contamination may be reduced by operating the present system in a sterile, or clean room environment. Closed housings may also enhance performance by reducing dust particles. In one embodiment, the surfaces of isolator 200 are electrostatically charged to repel any airborne or water-born dust particles.

Furthermore, attachment and removal of antibodies, or other binding partners, from the silicone surface also may affect the surface characteristics of the silicone. Similar to water and dust, the biochemicals applied to the silicone surface may weaken or degrade the bonds between the surfaces. In an application having two bonded silicone surfaces forming isolator 200 and with antibodies placed only in the channel regions, such problems may not arise. Other structures to enhance the rigidity of the column may also be used.

One embodiment of detector 300 facilitates a higher fluid flow rate. For example, detector 300 includes an orifice having moveable side walls, thus enabling changes in the dimension of the orifice. Large microorganisms unable to pass the orifice can become trapped and create an obstruction to further testing. With a fixed orifice, removal of obstructions entails backflushing the channel. With a variable orifice, removal of obstructions may entail selection of a larger orifice dimension, thus enabling the obstructing microorganism to freely flow through the orifice. Micro-electro-mechanical system (MEMS) actuators having beams coated with metal may facilitate implementation of a variable orifice design. In one embodiment, the user controls the orifice dimension.

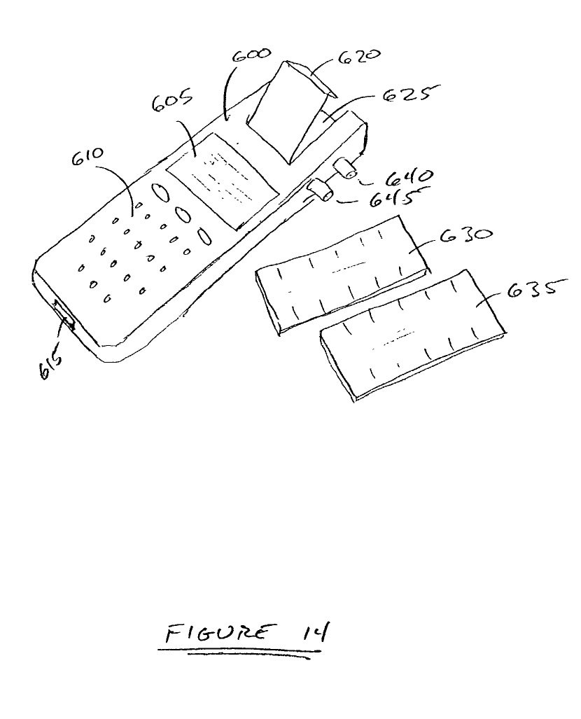

Integration of isolator 200 with detector 300, to form system 100, will further enhance detection and measurement of microorganisms. In one embodiment, isolator 200 and detector 300 are integrated into a portable, battery operated handheld device 600, a perspective view of which is illustrated in FIG. 14. Device 600 includes a visual display 605 and a plurality of controls 610. Connector 615 provides electrical terminals for coupling external devices, such as memory devices, visual displays, or other communication devices. Device 600 includes cartridge bay 625, under openable lid 620, for receiving a cartridge 630 or 635. More than one cartridge 630 or 635 may be simultaneously received by cartridge bay 625. Coupler 640 receives a fluid sample and coupler 645 discharges the bypass fluid of the sample. Other means of storing the isolated and detected microorganisms may be provided with an internal or external storage vessel. Device 600 may include an internal pump for moving the fluid sample through system 100. Disposable modules 630 and 635 include isolator 200 having the isolation element as well as a microsensor detection system. The portable device 600 tallies and determines viability of organisms passing through a plurality of passageways, or channels. An operator may select a particular cartridge, 630 or 635, based on the properties and parameters of the test environment and the targeted substance. For example, with regard to ground water testing for C. parvum, cartridge 630 having a particular isolator and detector module may be selected, whereas when testing fruit juices, cartridge 635 having a different isolator and detector module may be selected.

Different binding partners may also be selected depending upon the targeted substance being monitored. An earlier example described isolation and detection of C. parvum oocysts. As another example, isolator 200 may be adapted to filter and isolate a target chemical or biological substance. In one embodiment, isolator 200 includes an immobilized binding partner on an interior surface of pathway 220A or 220B. The immobilized binding partner, or biofilm may be selected to bind with a particular substance in a “lock and key” fashion. A portion of the interior surface of isolator 200 may be coated with a biofilm. The biofilm may be installed within isolator 200 by means of capillary action in a manner akin to the installation of the sample fluid. The biofilm binds with a particular target substance. In various embodiments, the biofilm may bind with one or more targeted substances.

The biofilm, having one or more binding partners, may be selected to bind to a desired target substance, or substances, wherein said bound target substance, or substances, is then later eluted and passed to detector 300. For example, one protein (such as an antibody) may be used as a binding partner for purposes of detecting a second protein (such as an antigen). By way of example only, and not by way of limitation, other pairs include using a receptor for detecting a ligand such as using a cellular receptor to identify a ligand that binds to such receptor, using a protein for detecting a peptide, using a protein for detecting a DNA, using a first DNA sequence to detect a second DNA sequence, using a metallic ion to detect a chelator, and using an antibody, or an antibody fragment, for detecting an antigen or analyte.

It will be recognized that the aforementioned examples bind to each other in a “lock and key” fashion by ionic bonding, covalent bonding or a combination thereof. In some cases, the binding partner may bind specifically to a single target substance or subunit thereof. Consequently, either the “lock” can be immobilized in isolator 200 for detecting the “key” or the “key” can be immobilized in isolator 200 for detecting the “lock.” As an example, a peptide may be the binding partner in isolator 200 for use in detecting a protein. The binding partner immobilized in isolator 200 can be DNA and thus, the present system is responsive to the substantial DNA complement. The bound, or “hybridized” DNA sequences can then be treated or “washed” under various conditions of stringency so that only DNA sequences that are highly complementary (e.g., that has high sequence identity) will be retained in isolator 200.

The binding partner can also bind to a plurality of substances, in which case, isolator 200 will filter and concentrate any substance binding to isolator 200. In addition, more than one binding partner may be immobilized in a particular isolator 200 to enable filtration of multiple molecules. Multiple binding partners may be immobilized in the same or different regions of isolator 200.

The binding partner can include an antibody for detection of an antigen, or binding partner includes an antigen for detection of an antibody. Examples of antigens include proteins, oligopeptides, polypeptides, viruses and bacteria. For instance, antigens include OMPₐ, OMP_{b} and OMP_{c}, commonly referred to as outer membrane protein “a” “b” and “c.” In such cases involving antigens, the interaction includes one or more amino acid interactions wherein the amino acids are spatially arranged to form two complementary surfaces in three dimensions. Each surface includes one or more amino acid side chains or backbones.

The binding partner can include an antibody for detection of a hapten, or the binding partner can include a hapten for detection of an antibody. Haptens tend to be much smaller than antigens and include such compounds as transition metal chelators, multi-ring phenols, lipids and phospholipids. In such cases involving haptens, the interaction includes an intermolecular reaction of a surface of the hapten with one or more amino acids of the antibody, wherein the amino acids of the antibody are spatially arranged to form a complementary surface to that of the hapten.

The interaction between amino acids, such as antibody-antigen or antibody-hapten, arises by van der Waal forces, Lennard-Jones forces, electrostatic forces or hydrogen bonding. Consequently, immobilized binding partner interacts with the targeted substance in a manner beyond that of simple absorption of analyte into a matrix of some type. The interaction of binding partner with the target substance is characterized by rapid bonding, preferably bonding that is not reversible under ambient conditions, thus reducing the time required for reliable filtration using isolator 200.

Hybrid antibodies are also contemplated for either the target substance or binding partner. For example, a portion of a first antibody may be cleaved and a second antibody may be bonded to the remaining portion of the first antibody, thus forming a hybridized antibody. Such an antibody may subsequently bind with two forms of antigens or haptens. As yet another example, a third antibody may be bonded to the remaining portion of the first antibody, thus enabling subsequent bonding to additional antigens or haptens. The use of hybridized antibodies in isolator 200 yields a filter sensitive to multiple substances and may be desirable for certain applications where filtration of two or more analytes is desired.

The binding partner may be affixed, or immobilized, to isolator 200 using any of a number of techniques, including absorption, covalent bonding with or without linker or spacer molecules or complexation.

Releasing the microorganism from isolator 200 may entail any number of methods. For example, a buffered solution may be used to change the pH of the system, thus releasing the microorganism. In one embodiment, an electromagnetic field promotes the release of the microorganism.

In one embodiment, the channel of detector 300 includes a convergent section and a divergent section. Depending upon the sample fluid, the angle of convergence may be greater, or less than, the angle of divergence.

Various dielectric properties may be monitored. For example, in addition to the dielectric constant, it may be desirable to monitor dielectric loss, dielectric breakdown, dielectric strength, dielectric absorption, or other properties. Such measurements may indicate with greater reliability, the identity, and other parameters, concerning the contents of the orifice.

Electrodes at the orifice may have any number of configurations. For example, one embodiment provides that the first and second electrodes are interleaved, and thus the dielectric property of the substance, or subunit, in the orifice relies not on a field traversing a diameter, or other major dimension, of the orifice, but rather, on a dielectric property when measured at a bias.

Conclusion

Although specific embodiments have been illustrated and described herein, it will be appreciated by those of ordinary skill in the art that any arrangement which is calculated to achieve the same purpose may be substituted for the specific embodiment shown. This application is intended to cover any adaptations or variations of the present invention.

What is claimed is:

1. A microorganism detection system comprising:
a flow cell having a passageway including an antibody for said microorganism immobilized on an interior surface of said passageway, said passageway adapted to isolate said microorganism; and
a microchip sensor adapted to electronically detect a dielectric property of said isolated microorganism.

2. The system of claim 1 wherein said passageway is adapted to isolate a pathogenic microorganism.

3. The system of claim 1 wherein said passageway is adapted to isolate an oocyst.

4. The system of claim 1 wherein said passageway is adapted to isolate an oocyst of C. parvum bacterium.

5. The system of claim 1 further comprising a vessel adapted for retaining said isolated microorganism.

6. The system of claim 1 wherein said flow cell is adapted for isolating abiotic contaminants.

7. The system of claim 1 wherein said flow cell is adapted for isolating biotic contaminants.

8. The system of claim 1 wherein said microchip sensor is adapted for isolating abiotic contaminants.

9. The system of claim 1 wherein said microchip sensor is adapted for determining viability of said isolated microorganism.

10. The system of claim 1 wherein said microchip sensor is adapted for determining a dielectric constant of said isolated microorganism.

11. The system of claim 1 wherein said flow cell is adapted for concentrating said microorganism by capillary action.

12. The system of claim 1 wherein said binding partner includes an immobilized monoclonal antibody, polyclonal antibody, or binding fragment thereof, that binds to said microorganism.

13. The system of claim 1 wherein said binding partner includes an immobilized APTase, RNA APTase, or binding fragment thereof, that binds to said microorganism.

14. An apparatus for isolating a component of a fluid, said apparatus comprising:
an input port;
a first planar element having an input edge, a first output edge, and a first plurality of fluid pathways on a first surface of said first planar element between said first input edge and said first output edge, said first input edge of said planar element being coupled to said first input port;
a binding partner that selectively binds to said component, said binding partner immobilized on at least a subset of said first plurality of fluid pathways;
an output port coupled to said first output edge of said first planar element; and
a sealing surface sealably coupled to said first surface of said first planar element;
wherein fluid traversing said first planar element is communicated through said first plurality of said fluid pathways by said planar element and said sealing surface.

15. The apparatus of claim 14 wherein said first planar element comprises silicone elastomer.

16. The apparatus of claim 14 wherein said sealing surface comprises glass, an acrylic polymer, silicon or silicone.

17. The apparatus of claim 14 wherein said pathways of said first plurality of fluid pathways have a depth of about 20 to 40 μm.

18. The apparatus of claim 14 wherein said first planar element is hermetically bonded to said sealing surface.

19. The apparatus of claim 14 wherein said binding partner comprises an APTase.

20. The apparatus of claim 14 wherein said binding partner comprises a population of antibodies.

21. The apparatus of claim 14 wherein said first planar element performs affinity purification.

22. The apparatus of claim 14 wherein said first plurality of fluid pathways simulate porous media.

23. The apparatus of claim 14 wherein said first plurality of fluid pathways define a serpentine network of pathways.

24. The apparatus of claim 14 further comprising a second planar element having a second input edge, a second output edge, and a second plurality of fluid pathways on a first surface of said second planar element between said second input edge and said second output edge, said first surface of said second planar element coupled to said first surface of said first planar element and further wherein said binding partner is immobilized on at least a subset of said second plurality of fluid pathways and wherein fluid traversing said first planar element and said second planar element is communicated through said first plurality of fluid pathways and said second plurality of fluid pathways.

25. A method of manufacturing an isolator assembly, said method comprising:
receiving a micromachined mold having a plurality of pathways;
applying a silicone elastomer to said mold;
curing said silicone elastomer for a predetermined time;
removing said silicone elastomer from said mold;
immobilizing a binding partner for a predetermined microorganism to a portion of the silicone elastomer corresponding to the plurality of pathways of the mold; and
bonding a sealing surface to said silicone elastomer.

26. The method of claim 25 wherein applying a silicone elastomer to said mold comprises pouring a silicone elastomer over said mold.

27. The method of claim 25 wherein curing said silicone elastomer for a predetermined time comprises curing with heat.

28. The method of claim 25 wherein curing said silicone elastomer comprises curing by heating to a temperature between 55° C. and 65° C.

29. A microsensor comprising:
a fluid inlet for receiving a sample fluid including one or more discrete subunits;
a channel coupled to said fluid inlet, said channel including an interior surface coupled to an orifice, wherein said orifice is adapted for sequentially passing a single discrete subunit;
a first electrode coupled to said orifice;
a second electrode coupled to said orifice, said second electrode electrically isolated from said first electrode; and
a fluid outlet coupled to said channel;
wherein an electrical signal applied to said first electrode is capacitatively communicated to said second electrode as a function of said single discrete subunit within said orifice.

30. The microsensor of claim 29 wherein said channel includes a convergent section.

31. The microsensor of claim 29 wherein said channel includes a divergent section.

32. The microsensor of claim 29 wherein said first electrode and said second electrode are substantially planar.

33. The microsensor of claim 29 wherein said first electrode and said second electrode are substantially parallel.

34. The microsensor of claim 29 wherein said first electrode and said second electrode are fabricated of metal.

35. The microsensor of claim 29 wherein said first electrode and said second electrode are fabricated of gold.

36. The microsensor of claim 29 wherein said orifice has a first and second setting, wherein at a first setting, said orifice has a dimension greater than at a second setting.

37. The microsensor of claim 29 wherein said orifice is variable and subject to control by a user.

38. The microsensor of claim 29 wherein said first electrode and said second electrode generate an electric field within said orifice.

39. The microsensor of claim 29 wherein said orifice has a first side and a second side oriented opposite said first side and further wherein said first electrode is positioned on said first side and said second electrode is positioned on said second side.

40. A system for determining a dielectric property for a pathogen, said system comprising:
fluid intake means adapted to receive a sample fluid, said sample fluid including one or more discrete pathogen subunits, said fluid intake means adapted to receive a predetermined quantity of sample fluid;
first orifice means adapted for passing a single said discrete pathogen subunit, said first orifice means coupled to said fluid intake means;
electrode means adapted for generating an electric field in said first orifice means, said electric field transmitted through a sample fluid within said first orifice means;
signal generating means adapted for generating an input electrical signal and applying said input electrical signal to said electrode means; and
processor means adapted for receiving an output electric signal from said electrode means and determining a dielectric property of said subunit in said first orifice means based on said input electric signal and said output electric signal.

41. The system of claim 40 further comprising a fluid reservoir means coupled to said first orifice means and adapted for receiving said subunit from said first orifice means.

42. The system of claim 40 further comprising channel means coupled to said fluid intake means and said first orifice means, wherein said channel means further includes a convergent section.

43. The system of claim 42 wherein said channel means is adapted for isolating said single subunit of said pathogen.

44. The system of claim 40 wherein said signal generating means and said processor means are adapted for determining a dielectric constant, a dielectric loss, a dielectric breakdown voltage, a dielectric strength, or a dielectric absorption of said pathogen.

45. The system of claim 40 further comprising:
display means adapted for providing visual data to a human operator of said system, said display means coupled to said first orifice means, said electrode means, said signal generating means and said processor means;
user operable control means coupled to said first orifice means, said electrode means, said signal generating means, said processor means, said display means and said user operable control means adapted for facilitating interaction by said human operator;
battery means adapted for supplying power to said first orifice means, said electrode means, said signal generating means, said processor means, said display means, and said user operable control means;
housing means adapted for housing said first orifice means, said electrode means, said signal generating means, said processor means, said display means, said user operable control means and said battery means.

46. The system of claim 40 further comprising:
second orifice means adapted for passing a single subunit of said pathogen, said second orifice means coupled to said fluid intake means; and
second electrode means adapted for generating a second electric field in said second orifice means, said second electric field transmitted through a sample fluid within said second orifice means;
and further wherein said signal generating means are adapted for generating a second input electrical signal and applying said second input electrical signal to said second electrode means; and further wherein said processor means are adapted for receiving a second output electric signal from said second electrode means and determining a dielectric property of said single subunit of said pathogen in said second orifice means based on said second input electric signal and said second output electric signal.

47. The system of claim 40 further comprising a user-replaceable cartridge, wherein said cartridge carries said first orifice means and said electrode means.

48. A method of detecting a targeted substance, comprising:
introducing a sample fluid suspected of including said targeted substance to a capillary system having an interior surface, said interior surface including an immobilized binding partner that binds with a subunit of said target substance;
releasing said subunit from said immobilized binding partner;
exposing said released subunit to an electromagnetic field within an orifice, said orifice adapted for sequentially passing a single subunit; and
determining a dielectric property for said subunit in said orifice.

49. The method of claim 48 further comprising determining viability of said subunit based on said dielectric property.

50. The method of claim 48 further comprising retaining said subunit in a vessel.

51. The method of claim 48 wherein introducing a sample fluid includes introducing liquid water, liquid food, body fluid, or an atmospheric gas.
